(19) United States
(12) Patent Application Publication
(10) Pub. No.: US 2021/0407685 A1
(43) Pub. Date: Dec. 30, 2021

(54) SYSTEMS AND METHODS FOR DETERMINING INDICATORS OF RISK OF PATIENTS TO ADVERSE HEALTHCARE EVENTS AND PRESENTATION OF THE SAME

(71) Applicant: UNIVERSITY OF MARYLAND, BALTIMORE COUNTY, BALTIMORE, MD (US)

(72) Inventor: Ian STOCKWELL, Cooksville, MD (US)

(21) Appl. No.: 17/359,797

(22) Filed: Jun. 28, 2021

Related U.S. Application Data
(60) Provisional application No. 63/045,362, filed on Jun. 29, 2020.

(51) Int. Cl. G16H 50/30; G16H 50/50; G16H 50/20; G16H 10/60; G16H 40/20

(52) U.S. Cl. CPC G16H 50/30; G16H 50/50; G16H 40/20; G16H 10/60; G16H 50/20

(57) ABSTRACT

Systems and methods for determining indicators of risk of patients to adverse healthcare events and presentation of the same are disclosed. According to an aspect, a method includes receiving, from a database, data associated with a plurality of individuals. The method also includes correlating the data to an adverse healthcare event for one or more of the individuals. Further, the method includes generating a model that relates the data to the adverse healthcare event based on the correlation of the data to the adverse healthcare event. The method also includes applying the model to data of another individual to generate an indicator of risk of the other individual to the adverse healthcare event. Further, the method includes presenting the indicator of risk via a user interface.

### CROSS REFERENCE TO RELATED APPLICATION

This application claims priority to U.S. Provisional Patent Application No. 63/045,362, filed Jun. 29, 2020, and titled “Pre-AI” PREdicting Avoidable Institutionalizations, the content of which is incorporated herein by reference in its entirety.

### TECHNICAL FIELD

The presently disclosed subject matter relates generally to healthcare services and resources. Particularly, the presently disclosed subject matter relates to systems and methods for determining indicators of risk of patients to adverse healthcare events and presentation of the same.

### BACKGROUND

With regard to healthcare services, there is a limited number of healthcare facilities and resources available to a growing population needing such services. Many healthcare facilities face problems associated with managing capacity and resources as well as the flow of patients through the healthcare facilities. Also, there will be further demands as the aging population continues to grow. To adequately meet these demands, the capacity of healthcare services must either grow or become more efficient.

The management of healthcare services has become more efficient due to the use of computers to handle patient records, to manage the care of patients, and to manage healthcare resources. As result, the healthcare service capacity has improved. These computer systems must also assure that high quality services are provided while improving efficiency and capacity.

In view of the foregoing, there is a continuing need to improve the efficiency and capacity of healthcare services. Also, there is a continuing need to improve or at least maintain a high level of quality of healthcare services.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is a block diagram of a system configured to determine and present risk indicators of patients to adverse healthcare events in accordance with embodiments of the present disclosure;

FIG. 2 is a flow diagram of an example method for determining and presenting risk indicators of patients to adverse healthcare events in accordance with embodiments of the present disclosure;

FIG. 3 is a flow diagram for generating a model and applying a model in accordance with embodiments of the present disclosure; and

FIG. 4 is a flow diagram for a predictive algorithm workflow in accordance with embodiments of the present disclosure.

### SUMMARY

The presently disclosed subject matter relates to systems and methods for determining indicators of risk of patients to adverse healthcare events and presentation of the same. According to an aspect, a method includes receiving, from a database, data associated with a plurality of individuals. The method also includes correlating the data to an adverse healthcare event for one or more of the individuals. Further, the method includes generating a model that relates the data to the adverse healthcare event based on the correlation of the data to the adverse healthcare event. The method also includes applying the model to data of another individual to generate an indicator of risk of the other individual to the adverse healthcare event. Further, the method includes presenting the indicator of risk via a user interface.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

“About” is used to provide flexibility to a numerical endpoint by providing that a given value may be “slightly above” or “slightly below” the endpoint without affecting the desired result.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

Unless otherwise defined, all technical terms used herein have the same meaning as commonly understood by one of ordinary skill in the art to which this disclosure belongs.

As referred to herein, the terms “computing device” and “entities” should be broadly construed and should be understood to be interchangeable. They may include any type of computing device, for example, a server, a desktop computer, a laptop computer, a smartphone, a cell phone, a pager, a personal digital assistant (PDA, e.g., with GPRS NIC), a mobile computer with a smartphone client, or the like.

As referred to herein, a user interface is generally a system by which users interact with a computing device. A user interface can include an input for allowing users to manipulate a computing device, and can include an output for allowing the system to present information and/or data, indicate the effects of the user's manipulation, etc. An example of a user interface on a computing device (e.g., a mobile device) includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. A user interface can include an input for allowing users to manipulate a computing device, and can include an output for allowing the computing device to present information and/or data, indicate the effects of the user's manipulation, etc. An example of a user interface on a computing device includes a graphical user interface (GUI) that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object. For example, the user can use a track ball or arrow keys for moving a cursor to highlight and select the display object.

The display object can be displayed on a display screen of a mobile device and can be selected by and interacted with by a user using the interface. In an example, the display of the mobile device can be a touch screen, which can display the display icon. The user can depress the area of the display screen at which the display icon is displayed for selecting the display icon. In another example, the user can use any other suitable interface of a mobile device, such as a keypad, to select the display icon or display object. For example, the user can use a track ball or times program instructions thereon for causing a processor to carry out aspects of the present disclosure.

As used herein, the term “memory” is generally a storage device of a computing device. Examples include, but are not limited to, read-only memory (ROM) and random access memory (RAM).

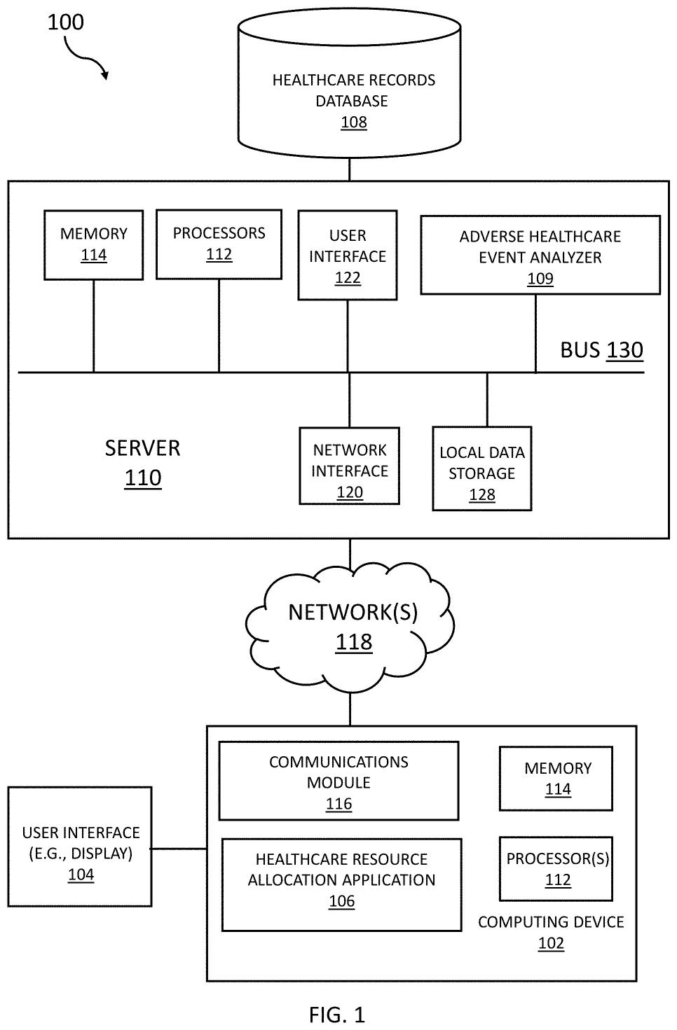

FIG. 1 illustrates a block diagram of a system 100 configured to determine and present risk indicators of patients to adverse healthcare events in accordance with embodiments of the present disclosure. Referring to FIG. 1, the system 100 includes a computing device 102. The computing device 102 may be a desktop computer, laptop computer, smartphone, or the like. The computing device 102 includes a user interface 104 for presentation of indicators of risk of individuals to adverse healthcare events in accordance with embodiments of the present disclosure. The user interface 104 in this example is a display that can be used to display a suitable representation (e.g., number or graphic) of an indicator of risk of one or more individuals to an adverse healthcare event. Alternative to a display, the user interface 104 may be any other suitable electronic device integrated into the computing device 102 or operably connected to the computing device 102 for presenting a representation of an indicator of risk. For example, in the alternative the user interface 104 may be a speaker or printer. In addition, the computing device 102 may include other user interfaces for receipt of user input, such as a keyboard, a mouse, microphone, or the like.

The computing device 102 includes a healthcare resource allocation application 106 configured to correlate data associated with multiple individuals (e.g., patients) to an adverse healthcare event for one or more of the individuals. The healthcare resource allocation application 106 is also configured to receive an indicator of risk and to present the indicator of risk to an operator. The healthcare resource allocation application 106 can use the user interface 104 to display the generated indicator of risk to the computing device's 102 operator. In accordance with embodiments, a server 110 can generate a model that relates the data to the adverse healthcare event based on the correlation of the data to the adverse healthcare event, and to apply the model to data of another individual to generate an indicator of risk of the other individual to the adverse healthcare event. Further, the generated model may be a regression model or any other suitable model for use in analyzing the individuals' data by suitable statistical processes for estimating a relationship between the data and an identified adverse healthcare event for one or more of the individuals. An example regression model is a linear regression model.

The server 110 can include a healthcare resource allocation analyzer 109 that can correlate data to an adverse healthcare event for one or more of the individuals, generate a model that relates the data to the adverse healthcare event based on the correlation of the data to the adverse healthcare event, and apply the model to data of another individual to generate an indicator of risk of the other individual to the adverse healthcare event. The analyzer 109 may be implemented by hardware, software, firmware or combinations thereof on the server 110. For example, the analyzer 109 may be implemented by one or more processors 112 and memory 114 of the server 110. The memory 114 may store instructions implemented by the processor(s) 112 for implementing functionality of the analyzer 109.

As an example, the application 106 may be configured to control a display to present to an operator an interface for interacting with the analyzer 106. The operator may use the application 106 for assessing one or more individual's risk of an adverse event such as, but not limited to, institutionalization admission, hospital admission, and death. The operator may interact with the application 106 to suitably identify the one or more individuals. Subsequent to receiving input of the identifier(s) for the individual(s), the application 106 can generate indicator(s) of risk of the identified individual(s) to an adverse healthcare event. The operator may also interact with the application 106 to specify the adverse healthcare event. In an example, the application 106 can be used to assess an individual's risk of nursing home admission.

In accordance with embodiments, the analyzer 109 at the server 110 may generate a regression model that relates data of multiple patients to an adverse healthcare event (e.g., nursing home admission) based on a correlation of that data to the event. In the example of FIG. 1, the data may be stored remotely in a healthcare records database 108. Example data stored in the database 108 includes, but is not limited to, demographic information of individuals, homelife information of individuals, cognition information of individuals, daily living information of individuals, chronic conditions of individuals, geographic locations (e.g., zip code) associated with individuals, and avoidable healthcare-related outcome information associated with individuals. Other example data stored in the database 108 includes administrative claims data, in-person assessment data, and/or the like. The analyzer 109 at the server 110 may use some or all of the data to generate the regression model in accordance with embodiments of the present disclosure.

In an alternative example, the model may be generated at another computing device. In this example, the server 110 has access to the database 108. The other computing device may also include an adverse healthcare event analyzer configured to generate the model in accordance with embodiments of the present disclosure. Further, the analyzer at the other computing device may apply the model to data of another individual to generate an indicator of risk of the other individual to the adverse healthcare event in accordance with embodiments of the present disclosure. In this case, the other computing device may communicate the indicator of risk to the computing device 102 for presentation to its operator.

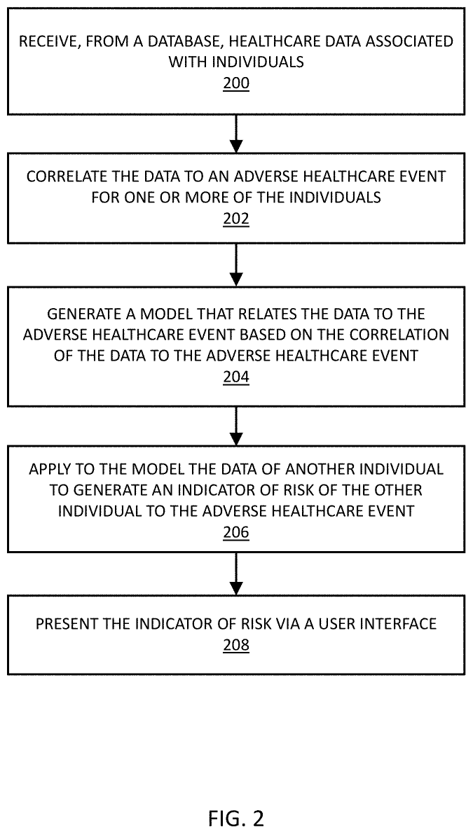

FIG. 2 illustrates a flow diagram of an example method for determining and presenting risk indicators of patients to adverse healthcare events in accordance with embodiments of the present disclosure. The method may be implemented by any suitable computing device or computing devices working together. With regard to FIG. 2, the method is described as being implement by the adverse healthcare event analyzer 109 of the server 110 and the application 106 of computing device 102.

Referring to FIG. 2, the method includes receiving 200, from a database, healthcare data associated with individuals. For example, the server 110 shown in FIG. 1 can identify healthcare records and request the identified healthcare records from the database 108. The server 110 may build a training dataset including demographic variables and clinical and functional indicators for a target population of individuals. For example these variables and indicators may be collected from a combination of administrative claims data and in-person assessment tools. It is noted that the functionality of the analyzer 109 may be implemented on one or more computing devices (e.g., one or more servers).

It is noted that the server 102 can include a user interface 122, memory 124, one or more processors 126, and local data storage 128 for implementing its functionality. These components of the server 102 may suitably communicate with each other via a bus 130. The server 102 may check a requester's credential for requesting the data. In response to approval of the credentials, the server 102 may be able to access the requested data.

Now turning again to FIG. 2, the method includes correlating 202 the data to an adverse healthcare event for one or more of the individuals. Continuing the aforementioned example, the analyzer 109 at the server 110 can correlate the received data to an adverse event for an individual. For example, the analyzer 109 can match the data to records for the adverse healthcare event (e.g., an avoidable outcome of interest such as nursing home admission, hospitalization, death, etc.) at a person level organized longitudinally.

The method of FIG. 2 includes generating 204 a model that relates the data to the adverse healthcare event based on the correlation of the data to the adverse healthcare event. Continuing the aforementioned example, the analyzer 109 can generate a proportional hazards regression model or other suitable model that relates the data to the adverse healthcare event based on the correlation of the data to the adverse healthcare event. The model can assign coefficient weights and significance scoring to each covariate.

The method of FIG. 2 includes applying 206 to the model the data of another individual to generate an indicator of risk of the other individual to the adverse healthcare event. Continuing the aforementioned example, the analyzer 109 can apply the generated model to another individual or several other individuals to generate an indicator of risk of the individual(s) to the adverse healthcare event. In an example, the analyzer 109 can use a Cox proportional hazards model.

The method of FIG. 2 includes presenting 208 the indicator of risk via a user interface. Continuing the aforementioned example, the server 110 may communicate to the computing device 102 the indicator of risk. For example, the computing device 102 can include a communications module 116 configured to send the request to the server 110 via one or more communications networks 118 (e.g., internet and/or local area networks). The server 110 can receive the request via its network interface 120. In response to receipt of the request, an analyzer 106 of the server 102 may communicate the indicator of risk to the computing device 102 via the network(s) 118. Further, the application 106 can control the user interface 104 to present the indicator of risk. For example, the user interface 104 can be display for displaying the indicator of risk. The indicator of risk can be used by a healthcare manager or other to assess risk and resources. Coefficients can be recalibrated with any significant policy, program, or population shift.

In an example use case, the systems and methods for determining indicators of risk of patients to adverse healthcare events disclosed herein can be used to assess the risk of nursing home admission for individuals applying to receive home- and community-based healthcare services. The training dataset of individual healthcare information can be built and matched to subsequent nursing home admissions as can be found in nursing home tracking data. Further, zip code level Census income data can be included as a proxy for broader community health impacts. The output can be an identification of the statistical significance and relative weight of each coefficient on nursing home admission. In accordance with embodiments, a regression model can be built based on this information for use in generating an indicator of risk of individuals to nursing home admission.

In another example use case, cluster analysis and/or classification trees can be used to group aggregate risk scores into groups (e.g., high risk, average risk, or low risk). Classification trees can be used for analyzing the data. Also, for example, cluster analysis can be used for continuous or high-dimensional categorical data.

In another example use case, potential outcomes measures can be expanded from nursing home admission information to include hospital use (avoidable versus unavoidable) and death. Further, models can be used for predicting hospital readmission, hospital transfer from a long-term care setting, violent injury, diabetic complications, and dementia complications.

In another example use case, risk indicators or scores can be used as an acuity-based rate adjuster in capitated payment systems or shared savings calculations in alternative payment models.

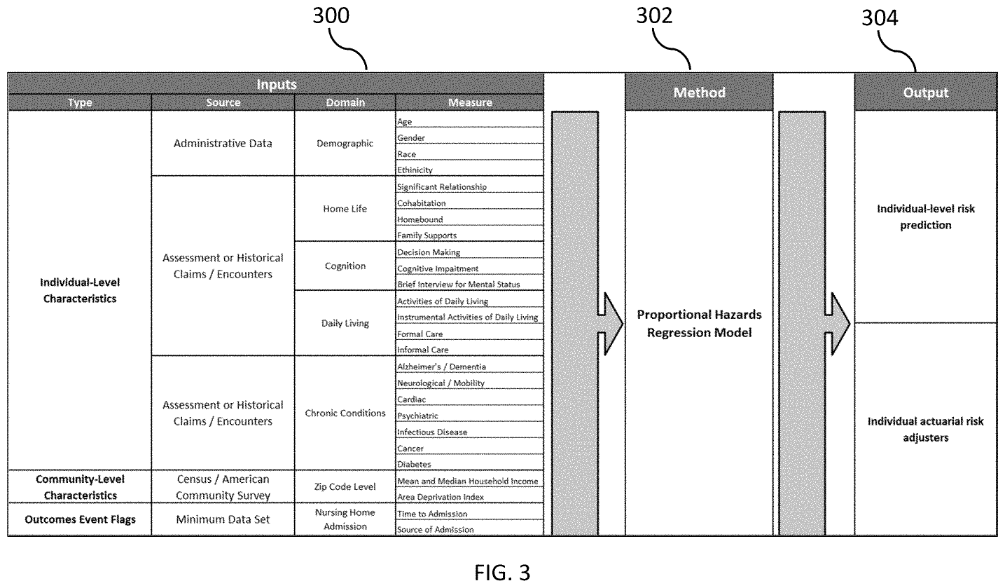

FIG. 3 illustrates a flow diagram for generating a model and applying a model in accordance with embodiments of the present disclosure. Referring to FIG. 3, inputs 300 include data associated with multiple individuals. The inputs 300 can include a type of input such as, but not limited to, individual-level characteristics and assessment or historical claims/encounters. Further, the inputs 300 can include community-level characteristic, such as Census and survey information. The inputs 300 also include outcome event flags, such as nursing home admission information (time to admission and source of admission).

With continuing reference to FIG. 3, this information can be used to generate a proportional hazards regression model 302 in accordance with embodiments of the present disclosure. The model 302 can be applied to an individual's healthcare data in accordance with embodiments of the present disclosure to produce an output 304 of an indicator of risk of an individual to nursing home admission. This can be an individual-level risk prediction. Further, the output 304 can include individual actuarial risk adjusters.

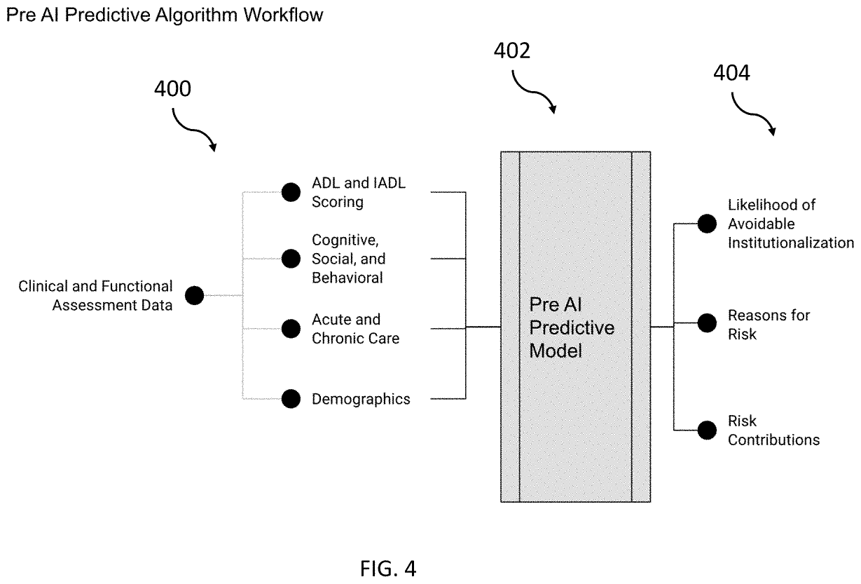

FIG. 4 illustrates a flow diagram for a predictive algorithm workflow in accordance with embodiments of the present disclosure. Referring to FIG. 4, steps of the process proceed from left to right. At step 400, clinical and functional assessment data is gathered. This data can include ADL and IADL scoring; cognitive, social, and behavioral data; acute and chronic care data; and demographics. This data used to generate a predictive model at step 402. Further, the model can be applied, at step 404, to an individual's data to generate information such as likelihood of avoidable institutionalization, and risk contributions.

The functional units described in this specification have been labeled as computing devices. A computing device may be implemented in programmable hardware devices such as processors, digital signal processors, central processing units, field programmable gate arrays, programmable array logic, programmable logic devices, cloud processing systems, or the like. The computing devices may also be implemented in software for execution by various types of processors. An identified device may include executable code and may, for instance, comprise one or more physical or logical blocks of computer instructions, which may, for instance, be organized as an object, procedure, function, or other construct. Nevertheless, the executable of an identified device need not be physically located together but may comprise disparate instructions stored in different locations which, when joined logically together, comprise the computing device and achieve the stated purpose of the computing device. In another example, a computing device may be a server or other computer located within a retail environment and communicatively connected to other computing devices (e.g., POS equipment or computers) for managing accounting, purchase transactions, and other processes within the retail environment. In another example, a computing device may be a mobile computing device such as, for example, but not limited to, a smartphone, a cell phone, a pager, a personal digital assistant (PDA), a mobile computer with a smartphone client, or the like. In another example, a computing device may be any type of wearable computer, such as a computer with a head-mounted display (HMD), or a smart watch or some other wearable smart device. Some of the computer sensing may be part of the fabric of the clothes the user is wearing. A computing device can also include any type of conventional computer, for example, a laptop computer or a tablet computer. A typical mobile computing device is a wireless data access-enabled device (e.g., an iPHONE® smartphone, a SAMSUNG® smartphone, an iPAD® device, smart watch, or the like) that is capable of sending and receiving data in a wireless manner using protocols like the Internet Protocol, or IP, and the wireless application protocol, or WAP. This allows users to access information via wireless devices, such as smart watches, smartphones, mobile phones, pagers, two-way radios, communicators, and the like. Wireless data access is supported by many wireless networks, including, but not limited to, Bluetooth, Near Field Communication, CDPD, CDMA, GSM, PDC, PHS, TDMA, FLEX, ReFLEX, iDEN, TETRA, DECT, DataTAC, Mobitex, EDGE and other 2G, 3G, 4G, 5G, and LTE technologies, and it operates with many handheld device operating systems, such as PalmOS, EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smartphone or smart watch that operates over GPRS (General Packet Radio Services), which is a data technology for GSM networks or operates over Near Field Communication e.g. Bluetooth. In addition to a conventional voice communication, a given mobile device can communicate with another such device via many different types of message transfer techniques, including Bluetooth, Near Field Communication, SMS (short message service), enhanced SMS (EMS), multi-media message (MMS), email WAP, paging, or other known or later-developed wireless data formats. Although many of the examples provided herein are implemented on smartphones, the examples may similarly be implemented on any suitable computing device, such as a computer.

An executable code of a computing device may be a single instruction, or many instructions, and may even be distributed over several different code segments, among different applications, and across several memory devices. Similarly, operational data may be identified and illustrated herein within the computing device, and may be embodied in any suitable form and organized within any suitable type of data structure. The operational data may be collected as a single data set, or may be distributed over different locations including over different storage devices, and may exist, at least partially, as electronic signals on a system or network.

The described features, structures, or characteristics may be combined in any suitable manner in one or more embodiments. In the following description, numerous specific details are provided, to provide a thorough understanding of embodiments of the disclosed subject matter. One skilled in the relevant art will recognize, however, that the disclosed subject matter can be practiced without one or more of the specific details, or with other methods, components, materials, etc. In other instances, well-known structures, materials, or operations are not shown or described in detail to avoid obscuring aspects of the disclosed subject matter.

The device or system for performing one or more operations on a memory of a computing device may be a software, hardware, firmware, or combination of these. The device or the system is further intended to include or otherwise cover all software or computer programs capable of performing the various heretofore-disclosed determinations, calculations, or the like for the disclosed purposes. For example, exemplary embodiments are intended to cover all software or computer programs capable of enabling processors to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed below.

In accordance with the exemplary embodiments, the disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl, or other suitable programming languages.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed is:

1. A method comprising:
receiving, from a database, data associated with a plurality of individuals;
correlating the data to an adverse healthcare event for one or more of the individuals;
generating a model that relates the data to the adverse healthcare event based on the correlation of the data to the adverse healthcare event;
applying the model to data of another individual to generate an indicator of risk of the other individual to the adverse healthcare event; and
presenting the indicator of risk via a user interface.

2. The method of claim 1, wherein the generated model is a regression model.

3. The method of claim 1, wherein the data associated with a plurality of individuals comprises one or more of demographic information of the individuals, homelife information of the individuals, cognition information of the individuals, daily living information of the individuals, chronic conditions of the individuals, geographic locations associated with the individuals, and avoidable healthcare-related outcome information associated with the individuals.

4. The method of claim 1, wherein the adverse healthcare event comprises institutionalization admission, hospital admission, and death.

5. The method of claim 1, wherein the adverse healthcare event comprises nursing home admission.

6. The method of claim 1, wherein the data in the database comprises administrative claims data and/or in-person assessment data.

7. The method of claim 1, wherein the steps of receiving, correlating, generating and apply are implemented at a first computing device comprising at least one processor and memory, and
wherein the step of presenting is implemented at a second computing device comprising the user interface.

8. The method of claim 7, further comprising communicating, by the first computing device, the indicator to the second computing device.

9. The method of claim 1, wherein the steps of receiving, correlating, generating and apply are implemented at a first computing device comprising at least one processor and memory, and
wherein receiving the data comprises receiving the data from a second computing device comprising memory including the database.

10. A system comprising:
a database comprising data associated with a plurality of individuals;
an adverse healthcare event analyzer configured to:
correlate the data to an adverse healthcare event for one or more of the individuals;
generate a model that relates the data to the adverse healthcare event based on the correlation of the data to the adverse healthcare event; and
apply the model to data of another individual to generate an indicator of risk of the other individual to the adverse healthcare event; and
a user interface configured to present the indicator of risk.

11. The system of claim 1, wherein the generated model is a regression model.

12. The system of claim 1, wherein the data associated with a plurality of individuals comprises one or more of demographic information of the individuals, homelife information of the individuals, cognition information of the individuals, daily living information of the individuals, chronic conditions of the individuals, geographic locations associated with the individuals, and avoidable healthcare-related outcome information associated with the individuals.

13. The system of claim 1, wherein the adverse healthcare event comprises institutionalization admission, hospital admission, and death.

14. The system of claim 1, wherein the adverse healthcare event comprises nursing home admission.

15. The system of claim 1, wherein the data in the database comprises administrative claims data and/or in-person assessment data.

16. The system of claim 1, wherein the functions of receiving, correlating, generating and apply are implemented at a first computing device comprising at least one processor and memory, and
wherein the function of presenting is implemented at a second computing device comprising the user interface.

17. The system of claim 16, wherein the first computing device is configured to communicate the indicator to the second computing device.

18. The system of claim 1, wherein the functions of receiving, correlating, generating and apply are implemented at a first computing device comprising at least one processor and memory, and
wherein the data is received at the first computing device from a second computing device comprising memory including the database.

19. A computer program product comprising a computer readable storage medium having program instructions embodied therewith, the program instructions executable by a computing device to cause the computing device to:
receive, at the computing device, data associated with a plurality of individuals;
correlate, at the computing device, the data to an adverse healthcare event for one or more of the individuals;
generate, at the computing device, a model that relates the data to the adverse healthcare event based on the correlation of the data to the adverse healthcare event;
apply, at the computing device, the model to data of another individual to generate an indicator of risk of the other individual to the adverse healthcare event; and
present the indicator of risk via a user interface.
